(19) United States
(12) Patent Application Publication
(10) Pub. No.: US 2012/0095330 A1
(43) Pub. Date: Apr. 19, 2012

(54) METHOD AND SYSTEM FOR POSITION DETERMINATION

(75) Inventors: Guy Shechter, Briarcliff Manor, NY (US); Douglas A. stanton, Ossining, NY (US)

(73) Assignee: KONINKLIJKE PHILIPS ELECTRONICS N.V., EINDHOVEN (NL)

(21) Appl. No.: 13/378,130

(22) PCT Filed: May 17, 2010
(86) PCT No.: PCT/IB2010/052173

§ 371 Date: Dec. 14, 2011

Related U.S. Application Data
(60) Provisional application No. 61/221,150, filed on Jun. 29, 2009.

(51) Int. Cl. A61B 5/05

(52) U.S. Cl. 600/424

(57) ABSTRACT

A system for tracking a device can include a medical device for placement in a target anatomy of a patient, one or more sensors in proximity to and connected to the medical device, wherein positioning data associated with the medical device is generated by the one or more accelerometric sensors, and a processor for determining at least one of a position and an orientation of the medical device based on the positioning data.

### CROSS REFERENCE TO RELATED APPLICATIONS

This application claims the benefit of U.S. provisional application Ser. No. 61/221,150, filed Jun. 29, 2009, (Applicant's docket no. PH013333US1) which is incorporated herein by reference. Related application is Ser. No. 61/221,138, “Method and Apparatus for Tracking in a Medical Procedure,” filed Jun. 29, 2009, (Applicant's docket no. PH013137US1).

The present application relates to the therapeutic arts, in particular to determining a position and/or orientation of a medical device and will be described with particular reference thereto.

A variety of systems and methods have been proposed to increase the precision of the placement of medical devices and instrumentalities into a particular target anatomy of a patient. Such systems include localization systems, which are used by a physician to visualize or ascertain the position and/or orientation of the medical instrument so as to enable the physician to accurately place the medical instrument in the anatomy of the patient. Current medical instrumentality localization systems often involve using electromagnetic sensing technologies, which require very expensive hardware and are susceptible to metal objects. This susceptibility to metal objects reduces the accuracy of measurements taken by the electromagnetic sensing technologies and any imaging data reliant on the measurements.

As a result, physicians placing a medical device into a patient based on electromagnetic sensing technologies and imaging data may have an incomplete or possibly inaccurate view of where the device and the anatomy are actually located with respect to one another. This can lead the physician to use an incorrect path when placing the device or even cause the physician to damage tissue unintentionally. Therefore, using a technology that is not necessarily reliant on the use of magnetic fields can decrease investment costs, while also increasing accuracy.

Accordingly, there is a need for a technique and system for determining the position and orientation of medical devices so as to enable more precise placement of the medical devices in a target anatomy during a medical procedure. There is a further need for a technique and system for enhancing imaging data with the determined position and orientation data.

This Summary is provided to comply with U.S. Rule 37 C.F.R. §1.73, requiring a summary of the invention briefly indicating the nature and substance of the invention. It is submitted with the understanding that it will not be used to interpret or limit the scope or meaning of the claims.

In accordance with one aspect of the exemplary embodiments, a method for tracking a medical device can include positioning the medical device into a target anatomy of a patient, wherein the medical device is in proximity to and is operably coupled to one or more accelerometric sensors; activating the one or more accelerometric sensors by providing energy to the one or more accelerometric sensors; receiving positioning data from the one or more accelerometric sensors; and determining at least one of a position and an orientation of the medical device based on the received positioning data.

In accordance with another aspect of the exemplary embodiments, a computer-readable storage medium can include computer-executable code stored therein, where the computer-executable code is configured to cause a computing device, in which the computer-readable storage medium is provided, to activate an accelerometric sensor in proximity to and integrally coupled to a medical device positioned in a target anatomy of a patient; receive positioning data from the activated accelerometric sensor; and determine at least one of a position and an orientation of the medical device based on the received positioning data.

In accordance with another aspect of the exemplary embodiments, a system for tracking a device can include a medical device for placement in a target anatomy of a patient; at least one accelerometric sensor in proximity to and connected to the medical device, wherein positioning data associated with the medical device is generated by the at least one accelerometric sensor; and a processor for determining at least one of a position and an orientation of the medical device based on the positioning data.

The exemplary embodiments described herein can have a number of advantages over contemporary systems and processes, including, but not limited to, providing robust imaging data and increased accuracy of medical device placement. Additionally, the system and method described herein can be utilized by retrofitting existing medical devices and is not susceptible to interference from metal objects. Still further advantages and benefits will become apparent to those of ordinary skill in the art upon reading and understanding the following detailed description.

The above-described and other features and advantages of the present disclosure will be appreciated and understood by those skilled in the art from the following detailed description, drawings, and appended claims.

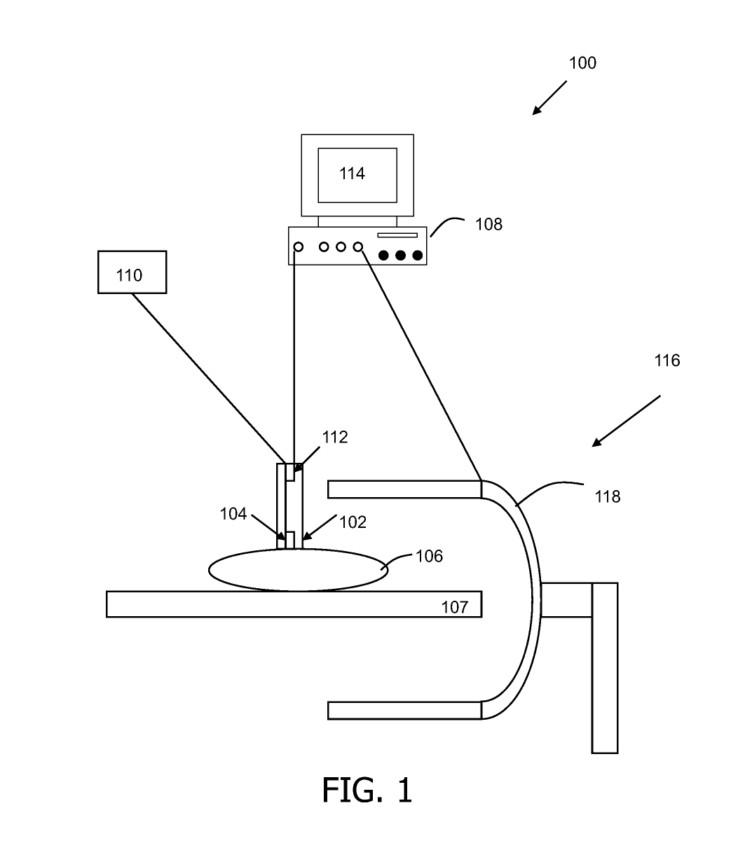

FIG. 1 is a schematic illustration of a system for determining the position and/or orientation of a medical device according to one exemplary embodiment;

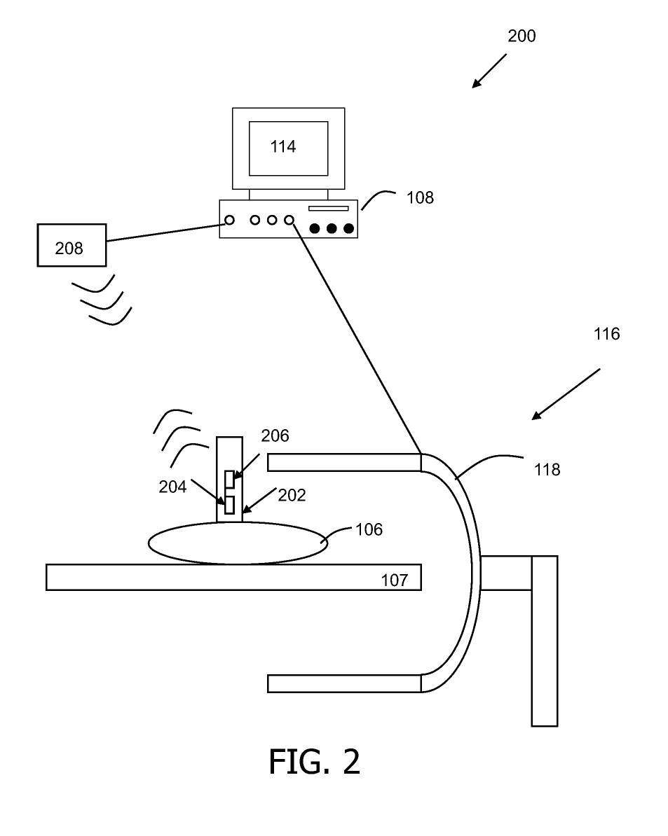

FIG. 2 is a schematic illustration of a system for determining the position and/or orientation of a medical device according to another exemplary embodiment;

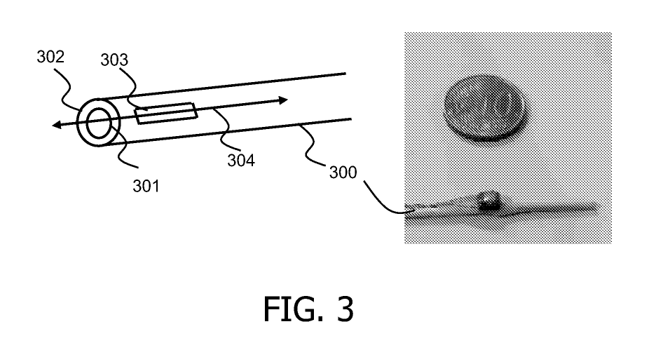

FIG. 3 is a schematic illustration of a medical device fitted with an accelerometric sensor for use with the system of FIGS. 1 and 2;

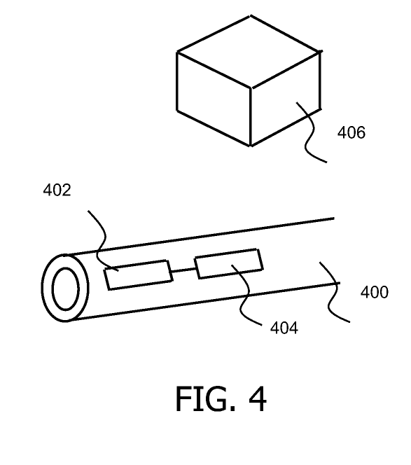

FIG. 4 is a schematic illustration of a medical device for use with the system of FIG. 2;

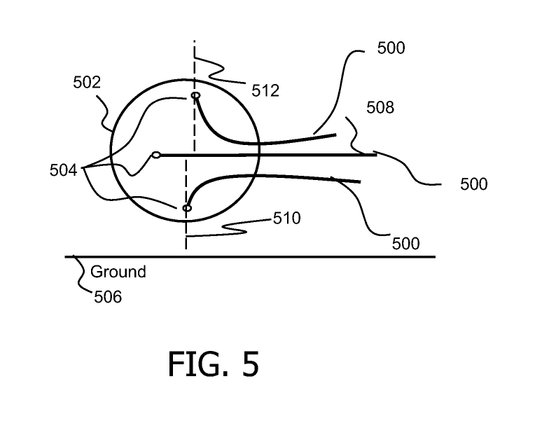

FIG. 5 is a schematic illustration of a medical device featured in various orientations in a target anatomy of a patient for use in the system of FIGS. 1 and 2;

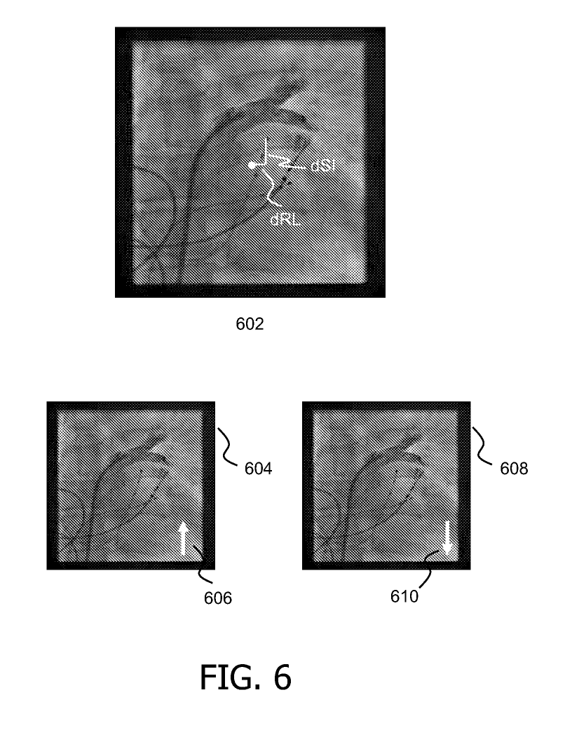

FIG. 6 is a schematic illustration of x-ray images generated in the system of FIGS. 1 and 2; and

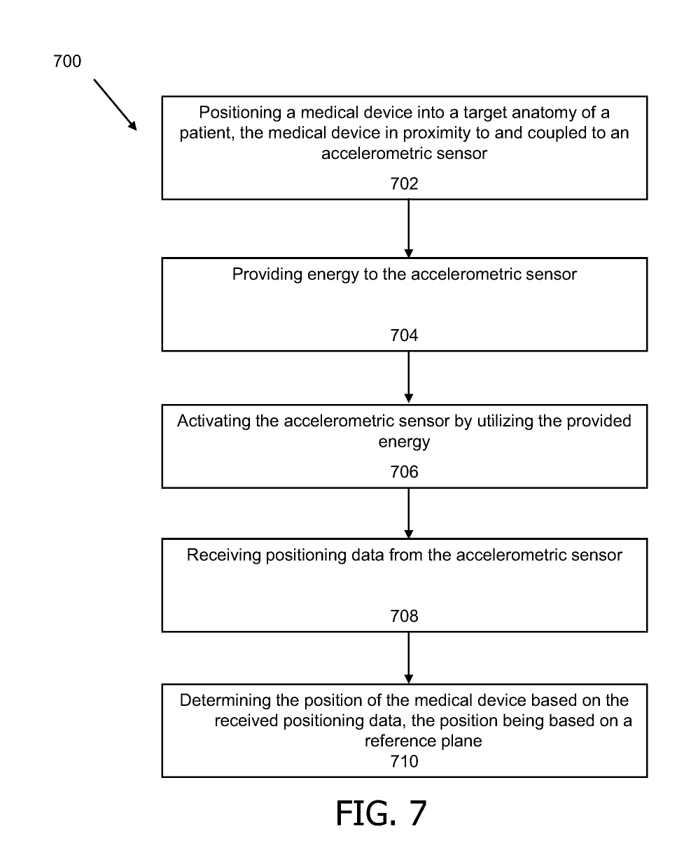

FIG. 7 is a method that can be used by the system and devices of FIGS. 1-6 for determining a position and/or orientation of a medical device during a medical procedure.

The exemplary embodiments of the present disclosure are described with respect to a system for tracking the position and/or orientation of a medical device to be utilized during a procedure for a human or animal. It should be understood by one of ordinary skill in the art that the exemplary embodiments of the present disclosure can be applied to, and utilized with, various types of medical or surgical devices, various types of procedures, and various portions of the body, whether the patient is a human or animal. The exemplary embodiments can also be used for enhancing an image, generated by an imaging device, of the medical device and target anatomy so as to indicate the orientation of the medical device with respect to the target anatomy. The exemplary embodiments are described herein as using accelerometric measurements in combination with images generated by imaging devices, but the present disclosure also contemplates determining the position and/or orientation of the medical device without utilizing imaging from imaging devices. Additionally, the exemplary embodiments described herein can operate without the use of magnetic fields or sources, however, the present disclosure also contemplates determining the position and/or orientation of the medical device using magnetic fields and/or sources. The use of the method and system of the exemplary embodiments of the present disclosure can be modified for other types of position determination.

Referring to FIG. 1, a system 100 for determining a position and/or orientation of a device is shown which can have a medical device 102, with a sensor 104 connected thereto. The medical device 102 can include a catheter, needle, probe, endoscope, or other medical device for entering a target anatomy 106 of a patient, who can be supported by support structure 107 such as a bed. The sensor 104 can be an accelerometric sensor or other sensor, such as a microelectromechanical (MEMS) device, capable of generating and providing position and/or orientation measurements, which can indicate a gravitational orientation toward or away from a reference plane, such as the earth. In one embodiment, accelerometric sensors can be used to determine position and/or orientation without having to utilize electromagnetic fields used in traditional localization systems. Therefore, the accelerometric sensors are not susceptible to interference from metal objects.

The sensor 104 can be located in proximity to and/or attached at various portions of the medical device 102, and can be fabricated during the production of the medical device 102. Additionally, the sensor 104 can be attached or placed on the outside of the medical device 102, the inner surface of the medical device 102, and/or embedded in the medical device 102 itself. For example, as shown in FIG. 1, the sensor 104 can be attached or proximately located at the tip or distal end of the medical device 102, however the sensor 104 can also be located elsewhere on the medical device 102. While the exemplary embodiment shows a single sensor 104, the present disclosure contemplates the use of multiple sensors 104 and multiple types of sensors capable of gathering positioning data.

The system 100 can also include a processor 108, which can be operably coupled to the sensor 104 wirelessly, through wires, or through other connection means. The processor 108 can be associated with a computer, personal digital assistant (PDA), mobile phone, communications device, or other computing device. Additionally, the system 100 can include a power supply 110, which can be connected to the medical device 102, and a signal acquisition device (SAD) 112. The power supply 110 can supply energy to the sensor 104 in order to activate the sensor 104. The SAD 112 can be coupled to the processor 108 and can be located and/or attached at a location on the medical device 102 that is opposite to the location of the sensor 104. However, the SAD 112 can be located elsewhere in the system 100 as well.

Operatively, a physician can position the medical device 102 and accompanying sensor 104 into the target anatomy 106 of the patient during a medical procedure. The target anatomy 106 can be a vessel, an organ, a tissue, or other part of the patient. The present disclosure can be utilized during various medical procedures, including percutaneous transluminal coronary angioplasty (PTCA), catheterization, cardiac angiography, vascular procedures, stenting, coil deployment, endoscopy procedures, electrophysiology procedures, x-ray based procedures, and any other procedure that can utilize the systems and devices in the disclosure. As the physician is positioning the medical device 102, the power supply 110 can supply power to the sensor 104 so as to enable the sensor 104 to generate measurement/positioning data relating to the position of the medical device 102. The measurement data can include, for example, acceleration data. When the sensor 104 captures the measurements, the sensor 104 can transmit the measurements to the SAD 112, which, in one embodiment, can process the measurements and create a position and/or orientation signal based on the measurements. The SAD 112 can then forward the position and/or orientation signal to the processor 108 for processing.

In another embodiment, the SAD 112 can transmit the measurements received from the sensor 104 and simply forward the raw measurements or data (such as a measured change in voltage) to the processor 108 without creating the position signal. Once the processor 108 receives the position and/or orientation signal or raw measurements, it can determine the position and/or orientation of the medical device 102 based on the received position and/or orientation signal or raw measurements. The determined position and/or orientation can be based on a reference plane, which can be horizontal, and the position and/or orientation can indicate a gravitation orientation toward or away from the reference plane. In one embodiment, the processor can obtain acceleration measurements from the position signal to calculate displacements of the medical device 102. In doing so, the determined position and/or orientation can identify a relative position offset to at least one of a previously determined position and orientation. The processor 108 can display the determined position via the display device 114.

In one embodiment, the system 100 can also include an imaging device 116, such as an x-ray machine 118, a computed tomography (CT) scanner, a positron emission tomography (PET) scanner, a magnetic resonance imaging (MRI) scanner, or other imaging device. The present disclosure contemplates the use of multiple imaging devices 116, which can be used in various combinations. The imaging device 116 can generate an image of the target anatomy 106 and the medical device 102 in the target anatomy 106, and can transmit the image to the processor 108. Once the image is received by the processor 108, the processor can store the image in a memory location and can overlay, superimpose, or otherwise combine the determined position and/or orientation of the medical device with the image. In another embodiment, the processor 108 can receive the image from the imaging device 116 and can create a new image including the determined position and/or orientation so as to add another dimension of data. The physician can view the image on the display device 114 and the determined position and/or images can be updated by the processor 108 in real-time so as to assist the physician during the course of the medical procedure.

Referring to FIG. 2, another exemplary embodiment of a system 200 for determining a position and/or orientation of a medical device is shown. The system 200 can include one or more components described with respect to system 100, including the support structure 107, processor 108, display device 114, imaging device 116, and scanner 118. System 200 can also include a medical device 202 operably coupled to and in proximity to a sensor 204, and a wireless energy transmission and/or reception device 206, such as an radio-frequency identification (RFID) tag. The sensor 204 can be an accelerometer and can be placed or attached on any surface of the medical device 202 or embedded in the medical device 202. The wireless device 206 can be operably coupled to the sensor 204 and can communicate with a transmitter 208. The transmitter 208 can be a RFID reader or other device capable of transmitting wireless signals, such as radio-frequency signals, to the wireless device 206. Additionally, the transmitter 208 can be in proximity to the medical device 202 and can be located outside of the patient.

Operatively, a physician can position the medical device 202 in a target anatomy of the patient. The transmitter 208 can transmit a wireless signal, such as a radio-frequency signal, which can be received by the wireless device 206. The radio-frequency signal can be utilized to energize the wireless device 206. Once energized, the wireless device 206 can store the energy and can activate the sensor 204 by providing energy, such as an input voltage and current, to the sensor 204. The activated sensor 204 can begin generating positioning data related to the position of the medical device 202. As positioning data is being generated by the sensor 204, the sensor 204 can transmit the data to the wireless device 206. The wireless device 206 can then transmit the positioning data to the transmitter 208, which can then forward the positioning data to the processor 108 for processing. In another embodiment, the sensor 202 and/or the wireless device 206 can send the positioning data to the processor 108. The processor 108 can proceed to determine the position and/or orientation of the medical device 202 based on the received positioning data. As mentioned above, the position and/or orientation of the medical device 202 can be based on a reference plane, such as a horizontal to the earth.

The system 200 can utilize imaging device 116 to generate an image of the target anatomy 106 and the medical device 202 in the target anatomy 106. The imaging device 116 can transmit the generated image to the processor 108, which can overlay, superimpose, or otherwise combine the determined position and/orientation of the medical device to the image. In another embodiment, the processor 108 can receive the image and/or imaging data from the imaging device 116 and create a new image, or otherwise adjust the presented image, to include the determined position and/or orientation of the medical device.

Referring to FIG. 3, a medical device 300 is shown that can be utilized with either of the systems 100 and 200. The medical device 300, such as the catheter shown, can include an inner diameter 301 and an outer diameter 302. Additionally, a sensor 303 can be attached anywhere on the inner surface, outer surface, and/or embedded in the wall of the medical device 300. The sensor 303 can be positioned such that the sensing axis of the sensor 303 is aligned with the longitudinal axis of the medical device 300 as illustratively shown in FIG. 3. Notably, the sensor 303 can be an accelerometer, which can be capable of measuring a gravitational orientation toward or away from a reference plane.

Referring to FIG. 4, a medical device 400 and a transmitter 406 is shown that can be utilized with either of the tracking systems 100 and 200. The medical devices 400 can include a sensor 402 that can be connected to a RFID device 404 or other similar device. The sensor 402 and the RFID device 404 can be placed near or in proximity to the tip or distal end of the medical device 400 as illustratively shown, or can be located elsewhere on or along the medical device 400. Much like the medical device of FIG. 3, the sensor 402 and the RFID device 404 can be aligned with the longitudinal axis of the medical device 400. The transmitter 406, which can be located outside of a target anatomy of the patient, can transmit a radio-frequency signal or other signal capable of energizing and activating the RFID device 404.

When the RFID device 404 receives a radio-frequency signal from the transmitter 406, the RFID device 404 can store the energy from the signal and can provide energy, such as an input voltage and current, to activate the sensor 402. Once activated, the sensor 402 can measure acceleration, such as based on gravity, and can generate positioning data relating to the position of the medical device 400. The positioning data can be transmitted to the RFID device 404, which can then transmit the data to the transmitter 406 or directly to processor 108 of systems 100 and/or 200. In another embodiment, the sensor 402 can directly transmit the data to the processor 108 so that the processor can determine the position and/or orientation of the medical device 400.

Referring to FIG. 5, a schematic illustration of a medical device for use in the system of FIGS. 1 and 2 is illustratively shown. The illustration depicts a medical device 500 in a target anatomy 502, such as a heart, of a patient. The medical device 500 can include a sensor 504, such as an accelerometer, that can be mounted on or in proximity to the tip or distal end of the medical device 500 or elsewhere. Additionally, a reference plane, which in this case is the ground 506, can also be utilized. As a physician positions the medical device 500 into the target anatomy 502 of the patient, the sensor 504 can receive energy, such as an input voltage and current, from a power source so as to activate the sensor 504. The sensor 504 can proceed to measure acceleration and generate positioning data.

For example, a power source can provide an input voltage of +Vs volts to the sensor 504. If the sensor 504 is in the horizontal position 508 with respect to the ground 506, the sensor 504 can generate an output voltage of +Vs/2. When the orientation/tilt of sensor 504 is substantially aligned with the vertical 510 towards the ground 506 and in the direction of the gravitational field, then the sensor 504 can generate an output voltage of zero volts. If the orientation/tilt of sensor 504 is substantially aligned with the vertical 512 away from the ground 506 and opposite the direction of the gravitation field, the sensor 504 can generate an output voltage of +Vs volts. These are only a sample of the possible measurements and output voltages generated by the sensor 504 and a range of voltages therebetween can be measured. As the orientation of the medical device 500 varies, so to can the corresponding output voltage generated by the sensor 504. The processor 108 can utilize the measurements generated by the sensor 504 to determine the position of the medical device 500 with respect to the target anatomy 502.

Referring to FIG. 6, a schematic illustration of x-ray images generated in the system of FIGS. 1 and 2. Image 602 depicts a two-dimensional anterior-posterior x-ray image. In this example, the position of the medical device in the right-left (RL) and superior-inferior (SI) patient axes can be measured. As shown in image 602, a point of reference, which, in this case, is chosen to be at the center of the image 602, can be utilized to calculate a measure of displacement (dRL, dSI). In one embodiment, the measurement can be made intuitively by the physician but can also be made automatically.

When the position and/or orientation data is received from the sensor, the processor can present the determined position and/or orientation to the physician in a variety of ways. For example, the position can be overlayed onto the x-ray image in the form of an arrow indicator as shown in images 604 and 608. The arrow indicator can be configured to point up to illustrate that the medical device is pointing away from the ground as shown in image 604. The arrow indicator can be configured to point down to illustrate that the medical device is pointing towards the ground as shown in image 608. In one embodiment, the size of the arrow can indicate the degree of tilt or orientation towards or away from the ground. For example, a large arrow that is pointing up can indicate that the medical device is oriented vertically away from the ground. As the medical device moves away from being oriented vertically and moves down towards the ground, the size of the arrow can scale down in magnitude. When the medical device becomes horizontal with respect to the ground, the arrow can disappear from the image. However, once the medical device starts to orient downwards from the horizontal a down pointing arrow can appear. The down pointing arrow can increase in size as the medical device becomes more and more vertically oriented towards the ground. The change in size of the arrow indicator can be proportional to the change in the orientation/tilt as measured by the sensor.

The use of up and down arrows that can change in magnitude is only one way to visually display the determined position to a physician. In another embodiment, text can be provided on the screen, which can state, for example, “the medical device is oriented horizontally,” “the medical device is oriented vertically,” or “the medical device is oriented towards the ground.” In yet another embodiment, the determined position and/or orientation can also be utilized to form a three-dimensional image. In still another embodiment, the determined position and/or orientation can be shown separately from the two-dimensional image provided by the x-ray machine or other scanning device.

Referring to FIG. 7, a method 700 for determining a position and/or orientation of a medical device during a medical procedure is shown. Method 700 can be employed for various types of medical treatments where positioning of a medical device is a desired criteria of the procedure. The steps in the method 700 can be incorporated in the systems of FIGS. 1 and 2. In step 702, the method 700 can include positioning a medical device into a target anatomy of a patient. The medical device can be in proximity to and can be operably coupled to one or more accelerometric sensors. The method 700 can also include providing the one or more accelerometric sensors with energy at step 704. In one embodiment, the energy can be provided to the accelerometric sensors by a power supply connected to the sensors through electrical wires. In another embodiment, the energy can be provided to the sensors through the use of radio frequency devices or other wireless remote power supplies that are operably coupled and in communication with the sensors.

In step 706, the method 700 can include activating the one or more accelerometric sensors by utilizing the provided energy. Once activated, the accelerometric sensors can generate positioning data associated with the medical device. The method 700 can further include receiving the generated positioning data from the one or more accelerometric sensors at step 708. For example, the sensors can transmit the positioning data to a signal acquisition device, which can then transmit the positioning data to a processor. As another example, the sensors can transmit data to a RFID device that can transmit the positioning data to a RFID reader or other similar device. The RFID reader can then transmit the data to a processor for processing. In step 710, the method 700 can include determining the position and/or orientation of the medical device based on the received positioning data. The position and/or orientation can be based on a reference plane such as the ground. One or more of the above steps can be performed by utilizing an electronic processor.

In one embodiment, the determined position and/or orientation of the medical device can identify a gravitational orientation towards or away from the reference plane. Additionally, the determined position and/or orientation of the medical device can identify a relative position offset to at least one of a previously determined position and orientation. In another embodiment, the method 700 can include generating an image of the target anatomy and the medical device in the target anatomy. The image can be generated by x-rays, computed tomography, positron emission tomography, magnetic resonance imaging, ultrasound imaging, or other types of imaging technologies. The determined position/orientation can be combined, overlayed, or superimposed onto the image to provide an enhanced and more detailed image to the physician. In yet another embodiment, the method 700 can include generating an image of the medical device using the determined position.

The invention, including the steps of the methodologies described above, can be realized in hardware, software, or a combination of hardware and software. The invention can be realized in a centralized fashion in one computer system, or in a distributed fashion where different elements are spread across several interconnected computer systems. Any kind of computer system or other apparatus adapted for carrying out the methods described herein is suited. A typical combination of hardware and software can be a general purpose computer system with a computer program that, when being loaded and executed, controls the computer system such that it carries out the methods described herein.

The invention, including the steps of the methodologies described above, can be embedded in a computer program product. The computer program product can comprise a computer-readable storage medium in which is embedded a computer program comprising computer-executable code for directing a computing device or computer-based system to perform the various procedures, processes and methods described herein. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed as the best mode contemplated for carrying out this invention, but that the invention will include all embodiments falling within the scope of the appended claims.

The Abstract of the Disclosure is provided to comply with U.S. Rule 37 C.F.R. §1.72(b), requiring an abstract that will allow the reader to quickly ascertain the nature of the technical disclosure. It is submitted with the understanding that it will not be used to interpret or limit the scope or meaning of the claims.

What is claimed is:

1. A method for tracking a medical device, the method comprising:
positioning the medical device (102) into a target anatomy (106) of a patient, wherein the medical device is in proximity to and is operably coupled to at least one accelerometric sensor (104);
activating the at least one accelerometric sensor by providing energy thereto;
receiving positioning data from the at least one accelerometric sensor; and
determining at least one of a position and an orientation of the medical device based on the received positioning data.

2. The method of claim 1, wherein the at least one of the determined position and orientation of the medical device (102) identifies a gravitational orientation toward or away from a reference plane.

3. The method of claim 1, wherein the at least one of the determined position and orientation of the medical device (102) identifies a relative position offset to at least one of a previously determined position and orientation.

4. The method of claim 1, further comprising generating an image of the target anatomy (106) and the medical device (102) in the target anatomy.

5. The method of claim 4, further comprising generating the image by utilizing at least one of x-rays, computed tomography, magnetic resonance imaging, and ultrasound imaging.

6. The method of claim 1, further comprising generating an image of the medical device (102) using the at least one of the determined position and orientation, wherein the image is based on at least one of x-ray, computed tomography, magnetic resonance imaging and ultrasound imaging.

7. The method of claim 2, wherein the reference plane of the at least one of the determined position and orientation is horizontal.

8. A computer-readable storage medium in which computer-executable code is stored, the computer-executable code configured to cause a computing device, in which the computer-readable storage medium is provided, to:
activate an accelerometric sensor (104) in proximity to and integrally coupled to a medical device (102) positioned in a target anatomy (106) of a patient;
receive positioning data from the activated accelerometric sensor; and
determine at least one of a position and an orientation of the medical device based on the received positioning data.

9. The computer-readable storage medium of claim 8, further comprising computer-executable code for causing the computing device to generate an image of the target anatomy (106) and the medical device (102) in the target anatomy.

10. The computer-readable storage medium of claim 9, further comprising computer-executable code for causing the computing device to generate the image by utilizing at least one of x-rays, computed tomography, magnetic resonance imaging, and ultrasound imaging.

11. The computer-readable storage medium of claim 9, further comprising computer-readable executable code for causing the computing device to generate the image using the at least one of the determined position and orientation.

12. The computer-readable storage medium of claim 9, further comprising computer-readable executable code for causing the computing device to provide an indicator on the image for indicating the at least one of the determined position and orientation, wherein the indicator points up when the at least one of the determined position and orientation is oriented away from a reference plane and down when the at least one of the determined position and orientation is oriented towards the reference plane.

13. The computer-readable storage medium of claim 12, wherein the at least one of the determined position and orientation of the medical device (102) identifies a gravitational orientation toward or away from the reference plane.

14. A system for tracking a device, the system comprising:
a medical device (102) for placement in a target anatomy (106) of a patient;
at least one accelerometric sensor (104) in proximity to and connected to the medical device, wherein positioning data associated with the medical device is generated by the at least one accelerometric sensor; and
a processor (108) for determining at least one of a position and an orientation of the medical device based on the positioning data.

15. The system of claim 14, further comprising a power supply (110) and a signal acquisition device (112) operably coupled to the at least one accelerometric sensor (104), wherein the power supply provides energy to activate the at least one accelerometric sensor, wherein the at least one activated accelerometric sensor transmits the positioning data to the signal acquisition device, and wherein the signal acquisition device transmits a position signal to the processor (108), wherein the position signal is based on the positioning data.

16. The system of claim 14, further comprising a transmitter (208) in proximity to the medical device and located outside the patient, wherein the transmitter is operable to emit a wireless signal to energize a wireless device (206) located in proximity to the at least one accelerometric sensor.

17. The system of claim 16, wherein the energized wireless device (206) provides energy to the at least one accelerometric sensor so as to enable the at least one accelerometric sensor to generate the positioning data.

18. The system of claim 17, wherein the wireless device (206) receives the generated positioning data from the at least one accelerometric sensor and transmits the positioning data to the transmitter (208), wherein the transmitter transmits the positioning data to the processor (108) for determining the at least one of the position and the orientation.

19. The system of claim 14, further comprising an imaging device (116) for generating an image of the target anatomy and the medical device in the target anatomy.

20. The system of claim 19, wherein the at least one of the determined position and orientation is utilized to supplement the generated image.

21. The system of claim 14, wherein the at least one of the determined position and orientation of the medical device identifies a gravitational orientation toward or away from a reference plane.
